(19) United States
(12) Patent Application Publication
(10) Pub. No.: US 2018/0303730 A1
(43) Pub. Date: Oct. 25, 2018

(54) COMPOSITION HAVING A COLOUR TRANSFORMATION DURING APPLICATION

(71) Applicant: L'OREAL, Paris (FR)

(72) Inventors: Yangdong CHEN, Shanghai (CN); Thierry COTTON, Shanghai (CN)

(73) Assignee: L'OREAL, Paris (FR)

(21) Appl. No.: 15/779,321

(22) PCT Filed: Dec. 18, 2015
(86) PCT No.: PCT/CN2015/097865

§ 371 Date: May 25, 2018

(51) Int. Cl. A61K 8/34; A61Q 19/02; A61Q 19/00; A61K 8/31; A61K 8/44; A61K 8/46; A61K 8/60

(52) U.S. Cl. CPC A61K 8/342; A61Q 19/02; A61Q 19/001; A61K 8/31; A61K 8/604; A61K 8/44; A61K 8/442; A61K 8/463; A61K 8/347

(57) ABSTRACT

A composition comprising a specific combination of surfactants, greater than or equal to 10% by weight of C₁-C₄ alcohol; and at least one solid fatty alcohol, can be used in caring for keratin materials, in particular the skin and lips.

The present invention relates to a composition having a unique colour transformation during application. It relates more particularly to a composition for caring for keratin materials, with a combination of surfactants and fatty alcohols for delivering a visible colour transformation during application. The invention also relates to a process for caring for keratin materials and in particular skin, by applying the composition of the present invention on the keratin materials.

### PRIOR ART

It is always been an ultimate goal of the cosmetic filed to deliver to the consumers high efficient products with skin benefits such as hydration, moisturizing, whitening, cleansing, and so on.

Among all the compositions for caring for skin, masks are known to have high penetration efficacy on the skin. As a result, a two-digit market growth of mask cosmetic products in China was seen.

Conventionally, the masks are grouped to three main categories: tissue masks, the dominant and most developed category; leave-on masks without tissue, such as sleeping masks used at night; and rinse-off masks without tissue, such as the clay mask for cleansing of the skin. More recently, sheet mask, or capsule mask are known as emerging categories.

Efforts have been made nowadays to formulate compositions for caring for the skin, in particular masks for caring for the skin, rinse-off or leave-on, with or without tissue, with active ingredients, in order to bring to the skin cosmetic benefits as mentioned above. Among which, whitening has always been one of the most interesting benefits.

Many whitening active ingredients are known to date. For example, ascorbyl glucosides, 3-O-ethyl ascorbic acid, niacinamide, or hydroxylated diphenylmethane derivatives are known for their whitening effects in the cosmetic field, and are widely used in the cosmetic products such as masks.

The other goal of the cosmetic products, which is equivalently important as the goal mentioned above, is the usage quality. The compositions are favoured by the consumers when they are easily applied on the skin, presenting a pleasant skin sensory, or having a unique transformation during application. In particular, the transformation during application is gaining increasing attention of the consumers, thanks to the unique and visible change.

The transformation of colour during application, for example, from white and opaque to transparent, is not only a visible aesthetic aspect that is favoured by the consumers, but also an indication as well as motivation to the consumers to massage the skin for a period of time in order to facilitate the penetration of the cosmetic compositions. Consequently, the skin benefits brought by the active ingredients are enhanced.

However, none of the prior arts has disclosed a composition for caring for the skin with such a visible colour transformation.

On the other hand, surfactants with foaming properties are widely used for generating foam during application of a cosmetic product. They are mainly used in detergent or cleansing products. A transformation of colour is also observed while applying cosmetic products with these surfactants. However, the inventors found the result not satisfying.

On one hand, the time duration of the transformation turn to be so short that it does not provide the purpose of aesthetic aspect or indication as mentioned above, for example, the colour transformation may take less than 3 minutes (much less than the average time duration for application of a cosmetic product for caring for the skin, in particular masks).

On the other hand, when applying the products on the skin, the white colour occurred turned to be inhomogeneous. White traces and unevenness is observed.

There thus exists a need to formulate a composition for caring for keratin materials with an improved colour transformation from opaque to transparent.

Such an improvement requires a relatively long time duration, for example greater than or equal to 5 minutes, as well as a homogeneity spreading of the white colour on the skin.

There is also a need to formulate a composition as described above, with a good skin sensory, such as hydration and moisturizing.

Furthermore, there exists a need for formulating a composition as described above, with sufficient stability over time, and in particular under high temperatures, such as 45° C.

More preferably, the composition of the present invention, on top of all the effects mentioned above, brings to the skin benefits such as whitening effect.

### SUMMARY OF THE INVENTION

The applicant found that such a need can be met by formulating a composition with combination of surfactant(s), C₁-C₄ alcohol(s), and solid fatty alcohol(s).

More specifically, the invention relates to a composition comprising:

(a) less than or equal to 5% by weight of at least one anionic surfactant chosen from sulfates of alkyl, alkyl ether, alkylamido ether group containing 6 to 30 carbon atoms;
(b) less than or equal to 5% of at least one second surfactant, different from the surfactant (a), selected from the group consisting of betaine amphoteric surfactants, alkyl(poly)glycoside nonionic surfactants, or a mixture thereof; and
(c) greater than or equal to 10% by weight of C₁-C₄ alcohol; and
(d) at least one solid fatty alcohol,
the amounts being relative to the total weight of the composition.

The present invention also relates to a process for caring for keratin materials, in particular the skin and lips, comprising the application to the keratin materials of the composition according to the invention.

The invention also relates to the use of the composition according to the invention for caring for keratin materials, in particular the skin and lips.

According to the invention, “transparent” is determined by observing the skin in a period of time, after application of the composition of the invention on the skin.

More particularly, “transparent” means there is no white colour or trace on the skin, after application of the composition of the invention. The natural skin tone is observed.

By “keratin materials” we intend to mean human keratin materials and more specifically skin and lips.

By “skin sensory” we intend to mean a fresh, hydration, and moisturizing feeling on the skin after application of the composition of the invention.

“Stable over time” is understood to mean compositions of the present invention which, after storage at all temperatures between 4° C. and 45° C. for 2 months, do not exhibit any macroscopic change in colour, smell or viscosity, any variation in pH or any variation in microscopic appearance.

Other subjects and characteristics, aspects and advantages of the invention will emerge even more clearly on reading the description and the examples that follows.

In that which follows and unless otherwise indicated, the limits of a range of values are included within this range, in particular in the expressions “of between” and “ranging from . . . to . . . ”.

Moreover, the expression “at least one” used in the present description is equivalent to the expression “one or more”.

### DETAILED DESCRIPTION OF THE PRESENT INVENTION

### (a) Anionic Surfactant(s)

The composition of the present invention comprises less than or equal to 5% by weight of at least one anionic surfactant chosen from sulfates of alkyl, alkyl ether, alkylamido ether group containing 6 to 30 carbon atoms.

Anionic surfactant agent is understood to mean an amphiphilic compound in which the hydrophobic part carries an anionic hydrophilic group with a cationic counterion which is generally metallic (alkali metal, such as Na or K) or ammonium; the hydrophilic group is thus polar and capable of dissociating to give anions in aqueous solution.

More particularly the anionic part of the anionic surfactant according to the present invention is —OS(O)₂O—, and the anionic part comprising a cationic counter anion such as alkali or alkaline earth metal or organic cationic counter anion such as ammonium.

More particularly, the anionic surfactants according to the invention are chosen from: (C₆-C₃₀)alkyl sulfates, (C₆-C₃₀)alkyl ether sulfates, (C₆-C₃₀)alkylamido ether sulfates, or a mixture is used.

Optionally, the anionic surfactants comprises 1 to 4 alkylene oxide group.

More preferably, the anionic surfactants is chosen from sulfates of C₁₂-C₂₄ fatty alcohols, optionally containing 1 to 4 alkylene oxide groups, sulfates of C₁₂-C₂₄ fatty alcohols ethers, optionally containing 1 to 4 alkylene oxide groups, or a mixture thereof.

Mentions may be made of the anionic surfactants such as ammonium lauryl sulfate sold under the name Empicol® AL 30/FL3 by the company Huntsman, sodium lauryl sulfate sold under the name Texapon® LS 30 by the company BASF, sodium laureth sulfate containing in average 2.2 ethylene oxide groups that are sold by the company Cognis (BASF) under the name Texapon® AOS 225 UP, Rhodia under the name Rhodapex® esb-70/fla3, and Clariant under the name Genapol® LRO L′O, and sodium laureth sulfate containing in average 1 ethylene oxide group that is sold by the company Zhejiang Zanyu Technology under the name SLES (N1EO), and sodium cetearyl sulfate, sold by the company BASF under the tradename Lanette® E Granules.

Advantageously, the anionic surfactant is present in the composition in an amount ranging from 0.1% to 5% by weight, preferably from 0.5% to 3% by weight, relative to the total weight of the composition.

### (b) Second Surfactant(s)

The composition of the present invention comprises less than or equal to 5% by weight of at least one second surfactant, different from the surfactant (a), selected from the group consisting of betaine amphoteric surfactants, alkyl(poly)glycoside nonionic surfactants, or a mixture thereof.

Among the betaine amphoteric surfactants, mentions may be made in particular of (C₈-C₂₀)alkylbetaines, (C₈-C₂₀ alkyl)amido(C₂-C₈ alkyl)betaines, or a mixture thereof.

Among the (C₈-C₂₀)alkylbetaines, mentions may be made of behenylbetaine, cetyl betaine, cocoylbetaine, decylbetaine.

From alkylbetaines, cocoylbetaine is preferred, for example the products sold by the company Rhodia under the tradename Mirataine® BB/FLA, or the products sold by the company BASF under the tradename Dehyton® AB 30.

Among the optionally quaternized secondary or tertiary aliphatic amine derivatives that may be used, mention may also be made of the products of respective structures (A1) and (A2) below:

Rₐ—CON(Z)CH₂—(CH₂)ₘ—N⁺(R_{b})(R_{c})(CH₂COO⁻) (A1)

in which:
Rₐ represents a C₁₀-C₃₀ alkyl or alkenyl group derived from an acid Rₐ—COOH preferably present in hydrolysed coconut oil, a heptyl group, a nonyl group or an undecyl group,
R_{b} represents a β-hydroxyethyl group,
R_{c} represents a carboxymethyl group;
m is equal to 0, 1 or 2,
Z represents a hydrogen atom or a hydroxyethyl or carboxymethyl group;

Rₐ—CON(Z)CH₂—(CH₂)ₘ—N(B)(B′) (A2)

in which:
B represents —CH₂CH₂OX′, with X′ representing —CH₂—COOH, CH₂—COOZ′, —CH₂CH₂—COOH, —CH₂CH₂—COOZ′, or a hydrogen atom,
B′ represents —(CH₂)_{z}—Y′, with z=1 or 2, and Y′ representing —COOH, —COOZ′, —CH₂—CHOH—SO₃H or —CH₂—CHOH—SO₃Z′,
m′ is equal to 0, 1 or 2,
Z represents a hydrogen atom or a hydroxyethyl or carboxymethyl group,
Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane,
R_{a′} represents a C₁₀-C₃₀ alkyl or alkenyl group of an acid Rₐ—COOH preferably present in hydrolysed linseed oil or coconut oil, an alkyl group, in particular a C₁₇ alkyl group, and its iso form, or an unsaturated C₁₇ group.

The compounds corresponding to formula (A1) are preferred.

Among the compounds corresponding to formula (A1), mentions may be made of cocamidopropyl betaine, for example the product sold under the tradename Dehyton PK 45 by Cognis (BASF).

More preferably, the second surfactant (b) is chosen from cocamidopropyl betaine, cocoylbetaine, or a mixture thereof; preferably cocoylbetaine.

The term “alkyl(poly)glycoside” denotes an alkylpolyglycoside or an alkylmonoglycoside, also referred to in the present patent application as an alkylglycoside, which may be alkoxylated with one or more alkylene oxide groups, preferentially of C₂-C₄.

The alkyl(poly)glycoside nonionic surfactant(s) used, alone or as mixtures, in accordance with the present invention may be represented by formula (III) below:

R₇O—(R₈O)ₜ-(G)ᵥ (III)

in which:
R₇ represents a linear or branched, saturated or unsaturated alkyl group, containing from about 8 to 24 carbon atoms, or an alkyl phenyl group in which the linear or branched alkyl radical contains from 8 to 24 carbon atoms,
R₈ represents an alkylene group containing from about 2 to 4 carbon atoms,
G represents a saccharide unit containing 5 or 6 carbon atoms,
t denotes a value ranging from 0 to 10 and preferably from 0 to 4, and
v denotes a value ranging from 1 to 15.

Preferably, the alkyl(poly)glycoside nonionic surfactant(s) correspond to formula (III) in which:

R₇ denotes a linear or branched, saturated or unsaturated alkyl group containing from 8 to 18 carbon atoms,
G denotes glucose, fructose or galactose, preferably glucose,
t denotes a value ranging from 0 to 3, and is preferably equal to 0,
and R₈ and v are as defined previously.

The degree of polymerization of the alkyl(poly)glycoside nonionic surfactant(s) as represented, for example, by the index v in formula (IV) ranges on average from 1 to 15 and preferably from 1 to 4. This degree of polymerization more particularly ranges from 1 to 2 and better still from 1.1 to 1.5, on average.

The glycoside bonds between the saccharide units are 1,6- or 1,4-bonds; preferably 1,4-bonds.

The compounds of formula (III) that may be used in the present invention are especially represented by the products sold by the company Cognis under the names Plantaren® (600 CS/U, 1200 and 2000) or Plantacare® (818, 1200 and 2000). It is also possible to use the products sold by the company SEPPIC under the names Triton CG 110 (or Oramix CG 110) and Triton CG 312 (or Oramix NS 10), the products sold by the company BASF under the name Lutensol GD 70, or those sold by the company Chem Y under the name AG10 LK.

It is also possible, for example, to use the 1,4-(C₈-C₁₆)alkylpolyglucoside as an aqueous solution at 53% by weight relative to the total weight of the solution, sold by Cognis under the reference Plantacare® 818 UP.

Among all these alkyl(poly)glycoside nonionic surfactants, the decylglucoside (INCI: Decyl glucoside) sold by BASF under the reference Plantacare® 2000 UP is preferably used.

According to a preferred embodiment, the composition comprises a second surfactant (b), selected from the group consisting of (C₈-C₂₀)alkylbetaines, alkyl(poly)glycoside nonionic surfactant(s) of formula (III):

R₇O—(R₈O)ₜ-(G)ᵥ (III)

in which:
R₇ denotes a linear or branched, saturated or unsaturated alkyl group containing from 8 to 18 carbon atoms,
G denotes glucose, fructose or galactose, preferably glucose,
t denotes a value ranging from 0 to 3, and is preferably equal to 0,
R₈ represents an alkylene group containing from about 2 to 4 carbon atoms,
v denotes a value ranging from 1 to 15,
or a mixture thereof.

Preferably, the second surfactant (b) is present in the composition in an amount ranging from 0.01% to 5% by weight, in particular from 0.05% to 2% by weight, relative to the total weight of the composition.

(c) C₁-C₄ Alcohol(s)

The composition of the present invention comprises greater than or equal to 10% by weight of at least one C₁-C₄ alcohol.

A alcohol according to the present invention may preferably be chosen from linear or branched, saturated or unsaturated alcohols with at least one hydroxyl group, or dialkylene alcohols with at least one hydroxyl group, or a mixture thereof.

Mentions may be made of linear C₁-C₄ hydroxyalkyls, C₁-C₄ dialkylene alcohols, glycerin, or a mixture thereof.

Mentions may be made of ethanol, glycerol, propylene glycol, butylene glycol, diethylene glycol, glycerin, or a mixture thereof.

Preferably the alcohol of the present invention is glycerin. Mentions may be made of the glycerin which is commercially available under the tradename Glycerine 4810 from the company Oleon, or glycerine MIN USP/BP from the company loi Group Oleochemical.

In particular, the C₁-C₄ alcohol is present in the composition in an amount ranging from 10% to 70% by weight, more preferably from 15% to 50% by weight, relative to the total weight of the composition.

### (d) Solid Fatty Alcohol(s)

The composition of the present invention comprises at least one solid fatty alcohol.

The term “fatty alcohol” means a long-chain aliphatic alcohol comprising from 8 to 40 carbon atoms, preferably from 12 to 34 or even from 12 to 30 carbon atoms, and comprising at least one hydroxyl group OH. These fatty alcohols are neither oxyalkylenated nor glycerolated.

The “solid fatty alcohols” are solid at room temperature (25° C.) and at atmospheric pressure (780 mmHg or 1 atm.); they are water-insoluble, i.e. they have a solubility in water of less than 1% by mass and preferably less than 0.5% by weight.

Preferably, the solid fatty alcohols are of structure R—OH with R denoting a saturated or unsaturated, linear alkyl group, optionally substituted with one or more hydroxyl groups, comprising from 14 to 30 carbon atoms.

Preferably, the solid fatty alcohols used in the present invention are selected from fatty alcohols having from 14 to 30 carbon atoms.

Mentions may be made of, as solid fatty alcohols, myristyl alcohol (1-tetradecanol), cetyl alcohol (1-hexadecanol), palmitoleyl alcohol (cis-9-hexadecen-1-ol), stearyl alcohol (1-octadecanol), arachidyl alcohol (1-eicosanol), behenyl alcohol (1-docosanol), erucyl alcohol (cis-13-docosen-1-ol), lignoceryl alcohol (1-tetracosanol), ceryl alcohol (1-hexacosanol), myricyl alcohol, or melissyl alcohol (1-triacontanol).

More preferably, according to the present invention, the solid fatty alcohol is selected from alcohols having from 14 to 22 carbon atoms, such as cetyl alcohol (1-hexadecanol), stearyl alcohol (1-octadecanol), arachidyl alcohol (1-eicosanol), behenyl alcohol (1-docosanol), or a mixture thereof.

More particularly, the solid fatty alcohol are selected from cetyl alcohol (1-hexadecanol), stearyl alcohol (1-octadecanol), arachidyl alcohol (1-eicosanol), behenyl alcohol (1-docosanol), or a mixture thereof.

Mentions of solid fatty alcohols may be made of cetearyl alcohol sold under the name Lanette O Or by BASF, wherein containing predominantly a mixture of cetyl alcohol and stearyl alcohol, or behenyl alcohol sold under the name Lanette 22 by the company BASF, wherein containing predominantly a mixture of behenyl alcohol, arachidyl alcohol, and stearyl alcohol.

According to one embodiment, the solid fatty alcohol is present in the composition in an amount ranging from 0.05% to 10% by weight, preferably from 0.1% to 5% by weight, relative to the total weight of the composition.

According to a preferred embodiment, the present invention relates to a composition for caring for keratin materials, in particular the skin and lips, comprising:

(a) from 0.5% to 3% by weight of at least one anionic surfactant chosen from (C₆-C₃₀)alkyl sulfates;
(b) from 0.05% to 2% by weight of at least one second surfactant, different from the surfactant (a), selected from the group consisting of (C₈-C₂₀)alkylbetaines, alkyl(poly)glycoside nonionic surfactants, or a mixture thereof; and
(c) from 15% to 50% by weight of C₁-C₄ alcohol; and
(d) from 0.1% to 5% by weight of solid fatty alcohol,
the amounts being relative to the total amount of the composition.

### (e) Aqueous Phase

The composition of the invention further comprises at least one aqueous phase.

Preferably the aqueous phase is a continuous phase.

According to an embodiment, the aqueous phase includes water, as the case may be, in a mixture with water-soluble additives and/or solvents.

The aqueous phase may also comprise organic solvents miscible with water (at room temperature 25° C.) such as for example monoalcohols having from 2 to 6 carbon atoms, beside those claimed as ingredient (c) above, polyols notably having from 5 to 20 carbon atoms, preferably from 5 to 10 carbon atoms, atoms, such as pentylene glycol, hexylene glycol, caprylyl glycol, dipropylene glycol; glycol ethers (notably having from 3 to 16 carbon atoms) such as mono-, di- or tri-propylene glycol (C₁-C₄)alkyl ethers, mono-, di- or tri-ethylene glycol (C₁-C₄) alkyl ethers and mixtures thereof.

According to an embodiment, the aqueous phase of the compositions of the invention comprises a polyol notably caprylyl glycol.

The amount of aqueous phase may range, for example, from 0.1% to 99.9% by weight, preferably from 0.5% to 99% by weight relative to the total weight of the composition.

According to an embodiment, the amount of water in the compositions according to the invention ranges from 0.1% to 90% by weight, preferably from 5% to 80% by weight, more preferably from 30% to 75% by weight, relative to the total weight of the composition.

Preferably, the compositions of the invention comprise more than 50% by weight of water relative to the total weight of the composition.

The aqueous phase may also contain other additives such as water-soluble active ingredients, preservatives, salts, gelling agents, fillers, additional water-soluble or water-dispersible polymers, water-soluble dyes, and so on.

### (f) Oily Phase

The composition of the invention further comprises at least one oily phase.

Preferably the oily phase is a dispersed phase.

Preferably, the oily phase is present in the composition in an amount ranging from 0.01% to 50% by weight, relative to the total weight of the composition. The oily phase comprise at least one oil.

The term “oil” refers to any fatty body in liquid form at room temperature (20-25° C.) and atmospheric pressure. These oils may be of animal, plant, mineral or synthetic origin.

The oils may be volatile or non-volatile.

The term “volatile oil” refers to any non-aqueous medium capable of evaporating from the skin or lips, in less than one hour, at room temperature (20-25° C.) and atmospheric pressure (760 mmHg). The volatile oil is a volatile cosmetic oil, liquid at room temperature.

More specifically, a volatile oil has an evaporation rate of between 0.01 and 200 mg/cm²/min, inclusive.

The term “non-volatile oil” is intended to mean an oil remaining on the skin or keratin fiber at ambient temperature and atmospheric pressure. More specifically, a non-volatile oil has an evaporation rate strictly below 0.01 mg/cm²/min.

To measure this evaporation rate, 15 g of oil or a mixture of oils to be tested are introduced into a crystallizer, 7 cm in diameter, placed on a scale located in a large 0.3 m³ chamber temperature-controlled at a temperature of 25° C., and humidity-controlled with a relative humidity of 50%. The liquid is left to evaporate freely, without stirring, by providing ventilation with a fan (PAPST-MOTOREN, reference 8550 N, rotating at 2700 rpm) positioned vertically above the crystallizer containing the solvent, with the blades directed toward the crystallizer and at a distance of 20 cm from the base of the crystallizer. The mass of oil remaining in the crystallizer is measured at regular intervals. The evaporation rates are expressed in mg of oil evaporated per surface area unit (cm²) and per time unit (minute).

The oils that are suitable for the present invention may be hydrocarbon-based, silicone-based or fluorine-based.

According to the invention, the term “silicone oil” refers to an oil including at least one silicon atom, and in particular at least on Si—O group.

The term “fluorine oil” refers to an oil including at least one fluorine atom.

The term “hydrocarbon oil” refers to an oil containing primarily hydrogen and carbon atoms.

The oils may optionally include oxygen, nitrogen, sulfur and/or phosphorus atoms, for example, in the form of hydroxyl or acid radicals.

More preferably, the composition of the present invention comprises hydrocarbon oil(s).

Specifically, the volatile oils may be chosen from hydrocarbon oils having 8 to 16 carbon atoms, and in particular branched C₈-C₁₆ alkanes (also called isoparaffins or isoalkanes), such as isododecane (also called 2,2,4,4,6-pentamethylheptane), isodecane, isohexadecane, and, for example, the oils sold under the trade names ISOPARS® or PERMETHYLS®.

It is also possible to cite, as a hydrocarbon volatile oil, linear C₉-C₁₇ alkanes, such as dodecane (C₁₂) and tetradecane (C₁₄), sold respectively under the names PARAFOL® 12-97 and PARAFOL® 14-97 (Sasol), and, as alkanes obtained according to the method described in the international application WO 2007/068371 A1, such as the undecane (C₁₁) and tridecane (C₁₃) mixture sold under the name CETIOL® UT (Cognis).

The non-volatile oils may, in particular, be chosen from among the non-volatile hydrocarbon oils.

Preferably, examples of non-volatile hydrocarbon oil can be used in the present invention includes those containing in the structures at least on amide unit, liquid paraffin or derivatives thereof, squalane, isoeicosane, liquid petroleum jelly, naphthalene oil, or a mixture thereof.

The expression “oil containing in its structure at least one amide unit” will be understood throughout the text of the description to mean any compound comprising in its chemical structure at least one amide group (or function) of the type:

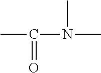

and simultaneously having the following characteristics:
a) liquid at 25° C.,
b) insoluble or immiscible in water at 25° C.,
c) no emulsifying properties.

Mentions may be made of such oils, for example, ethyl N-acetyl-N-butylaminopropionate, sold under the trade name Repellent R3535 by the company Merck; isopropyl N-lauroylsarcosinate, sold under the name Eldew® SL-205 by the company Ajinomoto; or N,N-diethyltoluamide, sold under the trade name Deet by the company Showa Denko.

Mentions may also be made of products such as squalane, which is commercially available under the tradename Plantasens® olive squalene sold by the company Clariant.

In a particular preferred embodiment, the composition further comprises at least one oil selected from squalene, isopropyl N-lauroylsarcosinate, or a mixture thereof.

According to an embodiment, the at least one oil (a) is present in an amount ranging from 0.01% to 5% by weight, and preferably from 0.05% to 2% by weight, relative to the total weight of the composition.

### (q) Additives

In a known manner, the composition of the invention may also contain one or more additives that are common in cosmetics or dermatology.

Examples of additives that may be mentioned include additional emulsifiers, active agents, antioxidants, fragrances, basic agents (triethanolamine, diethanolamine or sodium hydroxide) or acidic agents (citric acid), and also lipid vesicles or any other type of vector (nanocapsules, microcapsules, etc.), and mixtures thereof.

Mentions may be made to the active agents, such as whitening active ingredients. For example ascorbyl glucosides, 3-O-ethyl ascorbic acid, niacinamide, hydroxylated diphenylmethane derivatives.

As an example, it is possible to use hydroxylated diphenylmethane derivatives in the present invention. For example, it is possible to use 4-(1-phenylethyl)-1,3-benzenediol or 4-(1-phenylethyl)-1,3-dihydroxybenzene or otherwise called phenylethylresorcinol or phenylethylbenzenediol or styrylresorcinol. This compound has a CAS number 85-27-8, and is marketed under the name SYMWHITE 377® or BIO 377 by the company SYMRISE.

The compositions according to the invention may be, for example, in any of the galenical forms of O/W emulsions, for example in the form of a serum, a milk or a cream, and they are prepared according to the usual methods.

### (h) Method and Use

The composition of the present invention can be used for a process, such as a cosmetic process or method, for caring for the keratin materials, such as the skin and lips, in particular the face and the lips, by being applied to the skin, especially the face.

The present invention relates to a process for caring for keratin materials, comprising the application, to the surface of the said keratin materials, of at least one composition of the invention, wherein the keratin material is preferably skin and lips, in particular face.

Such a process contains a colour transformation from white to transparent.

The invention relates to a use of the composition of the present invention in caring for keratin materials, in particular skin and lips.

The examples that follow are aimed at illustrating the compositions and processes according to this invention, but are not in any way a limitation of the scope of the invention.

### Examples

### Example 1 Preparation of Invention and Comparative Formulas

Formulas according to the invention and outside of the invention (comparative) were prepared according to the concentration given in table 1.

TABLE 1
Invention formula A, B, C, and Comparative formula A′, B′

| | | | | | Comparative | |
|---|---|---|---|---|---|---|
| | | Invention formula | | | formula | |
| Phase | INCI name | A | B | C | A′ | B′ |
| A | DISODIUM | 0.1 | 0.1 | 0.1 | 0.1 | 0.1 |
| | EDTA | | | | | |
| B2 | PHENYLETHYL | 0.3 | 0.3 | 0.3 | 0.3 | 0 |
| | RESORCINOL | | | | | |
| B1 | CETEARYL | 7.0 | 7.0 | 7.0 | 7.0 | 7.0 |
| | ALCOHOL | | | | | |
| B1 | SQUALANE | 0.5 | 0 | 0 | 0 | 0 |
| | (Plantasens ® | | | | | |
| | olive squalene | | | | | |
| | from Clariant) | | | | | |
| B2 | ISOPROPYL | 0.5 | 0.5 | 0.5 | 0.5 | 0 |
| | LAUROYL | | | | | |
| | SARCOSINATE | | | | | |
| E | FRAGRANCE | 0.1 | 0.1 | 0.1 | 0.1 | 0.1 |
| A | PRESERVATIVE | 0.7 | 0.7 | 0.7 | 0.7 | 0.7 |
| A | WATER | QS 100 | QS 100 | QS 100 | QS 100 | QS 100 |
| B1 | GLYCERIN | 30 | 30 | 30 | 30 | 30 |
| | (Glycerine 4810 | | | | | |
| | from Oleon) | | | | | |
| A | CAPRYLYL | 0.2 | 0.3 | 0.3 | 0.3 | 0.3 |
| | GLYCOL | | | | | |
| A | SODIUM | 1.5 | 1.5 | 1.5 | 1.5 | 0.78 |
| | CETEARYL | | | | | |
| | SULFATE | | | | | |
| | (96% by active, | | | | | |
| | Lanette ® E | | | | | |
| | Granules from | | | | | |
| | BASF) | | | | | |
| A | COCO-BETAINE | 0.15 | 0 | 0.15 | 0 | 0 |
| | (Dehyton ® AB | | | | | |
| | 30 from BASF) | | | | | |
| A | DECYL | 0 | 0.25 | 0 | 0 | 0 |
| | GLUCOSIDE | | | | | |
| | (Plantacare ® | | | | | |
| | 2000 UP from | | | | | |
| | BASF) | | | | | |
| B1 | BEHENYL | 1 | 1 | 1 | 1 | 1 |
| | ALCOHOL | | | | | |
| | (Lanette 22 | | | | | |
| | from BASF) | | | | | |

Comparative formula A′ does not contain the second surfactant (b) as claimed;

Comparative formula B′ does not contain the second surfactant (b) as claimed.

The Invention and Comparative formulas were prepared following the steps of:

1. heat phase A to 50° C. for 10 min, stir until solubilizing;
2. heat phase B1 to 80° C. for 20 min until homogeneous;
3. heat phase B2 to 80° C. for 10 min, stir until solubilizing, then introduce phase B2 to phase B1, stirred for 5 min;
4. Introduce phase B(B1+B2) into phase A at 75° C., and turbine for 10 min, then cool down the mixture to 45° C., introduce phase C and turbine for 10 min, then stir and cool down to 25° C.;
5. introduce phase D and E respectively at room temperature 25° C., stir the mixture for 10 min.

Machine: the homogenizer VMI sold by the company i-Tech

### Example 2: Evaluation of the Invention and Comparative Formulas

The stability, colour transformation, and skin sensory of the Invention and Comparative formulas were evaluated.

The stability of the formulas were evaluated by leaving the formulas at 4° C., 25° C., and 45° C. for 2 months, and observing the formulas using microscope under polarized light.

The colour transformation was evaluation following the protocol of:

1. evenly spread 0.25 g of the invention and comparative formulas, respectively, on the clean back of the hand;
2. gently and evenly massage the hand with the formulas for 20 rounds, leaving the formulas on the hand;
3. observe the colour change from white to transparent in 15 mins,
4. rinse off the formulas from the hand.

The colour transformation effect was evaluated by time duration as well as a homogeneity spreading of the white colour on the skin.

For the purpose of the present invention, the time duration of the colour transformation is expected to be at least 5 minutes, preferably from 10 to 15 minutes.

The more homogenous the spreading of white colour in the skin, the better colour transformation effect of the formula presents.

The Invention and Comparative formulas were given to 10 consumers for application on the facial skin as facial masks, respectively, then they were rinsed off by water, and the skin sensory such as hydration, moisturizing were evaluated and scored by the consumers, respectively: 1, very poor; 2, poor; 3, acceptable; 4, very good; 5, just right, excellent sensory.

The results of the evaluation are as follow:

| | Invention formula | | | Comparative formula | |
|---|---|---|---|---|---|
| Item | A | B | C | A′ | B′ |
| Colour | Yes, excellent | Yes | Yes | No, obvious | No, too light, no |
| transformation/ | homogeneity | | | white trace | expected |
| homogeneity | of white | | | | obvious white |
| | colour during | | | | colour was |
| | application | | | | observed |
| Colour | totally | 15 min | 15 min | 15 min | 2 min |
| transformation/ | transparent in | | | | |
| time duration | 15 min | | | | |
| Stability | Stable | Stable | Stable | Stable | Stable |

All the formulas have hydration and moisturizing skin sensory.

It is observed that all formulas are stable.

Comparing to the comparative formulas A′ and B′, the invention formulas A, B, and C all have improved colour transformation, both in terms of a proper time duration, and homogeneity spreading of white colour.

1. A composition comprising:
(a) less than or equal to 5% by weight of at least one anionic surfactant selected from the group consisting of sulfates of alkyl, alkyl ether, and alkylamido ether group containing 6 to 30 carbon atoms;
(b) less than or equal to 5% of at least one second surfactant, different from the surfactant (a), selected from the group consisting of betaine amphoteric surfactants, alkyl(poly)glycoside nonionic surfactants, and mixtures thereof;
(c) greater than or equal to 10% by weight of C₁-C₄ alcohol; and
(d) at least one solid fatty alcohol,
the amounts being relative to a total weight of the composition.

2. The composition of claim 1, wherein the anionic surfactant is selected from the group consisting of (C₆-C₃₀)alkyl sulfates, (C₆-C₃₀)alkyl ether sulfates, (C₆-C₃₀)alkylamido ether sulfates, or a mixture and mixtures thereof.

3. The composition of claim 1, wherein the anionic surfactant (a) is present in the composition in an amount ranging from 0.1% to 5% by weight, relative to the total weight of the composition.

4. The composition of claim 1, wherein the betaine amphoteric surfactant is selected from the group consisting of (C₈-C₂₀)alkylbetaines, (C₈-C₂₀ alkyl)amido(C₂-C₈ alkyl)betaines, and mixtures thereof.

5. The composition of claim 1, wherein the alkyl(poly)glycoside nonionic surfactant is presented by the formula (III),
R₇O—(R₈O)ₜ-(G)ᵥ (III)
wherein:
R₇ represents a linear or branched, saturated or unsaturated alkyl group, containing from about 8 to 24 carbon atoms, or an alkyl phenyl group in which the linear or branched alkyl radical contains from 8 to 24 carbon atoms,
R₈ represents an alkylene group containing from about 2 to 4 carbon atoms,
G represents a saccharide unit containing 5 or 6 carbon atoms,
t denotes a value ranging from 0 to 10, and
v denotes a value ranging from 1 to 15;
wherein in formula (III):
R₇ denotes a linear or branched, saturated or unsaturated alkyl group containing from 8 to 18 carbon atoms,
G denotes glucose, fructose or galactose, preferably glucose, and
t denotes a value ranging from 0 to 3.

6. The composition of claim 1, wherein the alkyl(poly)glycoside nonionic surfactant is decylglucoside.

7. The composition of claim 1, wherein the second surfactant (b) is present in the composition in an amount ranging from 0.01% to 5% by weight, relative to the total weight of the composition.

8. The composition of claim 1, wherein the C₁-C₄ alcohol is selected from the group consisting of ethanol, glycerol, propylene glycol, butylene glycol, diethylene glycol, glycerin, and mixtures thereof.

9. The composition according to claim 1, wherein the C₁-C₄ alcohol is present in the composition in an amount ranging from 10% to 70% by weight, relative to the total weight of the composition.

10. The composition of claim 1, wherein the solid fatty alcohol (d) is selected from a fatty alcohol having from 14 to 30 carbon atoms.

11. The composition of claim 1, wherein the solid fatty alcohol is present in the composition in an amount ranging from 0.05% to 10% by weight, relative to the total weight of the composition.

12. A composition for keratin materials, comprising:
(a) from 0.5% to 3% by weight of at least one anionic surfactant chosen from (C₆-C₃₀)alkyl sulfates;
(b) from 0.05% to 2% by weight of at least one second surfactant, different from the surfactant (a), selected from the group consisting of (C₈-C₂₀)alkylbetaines, alkyl(poly)glycoside nonionic surfactants, and mixtures thereof;
(c) from 15% to 50% by weight of a C₁-C₄ alcohol; and
(d) from 0.1% to 5% by weight of a solid fatty alcohol,
the amounts being relative to the total a total amount of the composition.

13. The composition of claim 1, further comprising at least one oil selected from non-volatile hydrocarbon oils.

14. A process for caring for keratin materials, comprising applying the composition of claim 1 on the keratin materials.

15. The process of claim 14, wherein the composition is applied to skin and lips as the keratin materials.
